(19) United States
(12) Patent Application Publication
(10) Pub. No.: US 2024/0191166 A1
(43) Pub. Date: Jun. 13, 2024

(54) METHOD FOR PRODUCTION OF A RENEWABLE BIO-NATURAL GAS

(71) Applicant: TECHNICAL INFORMATION, LLC, Spokane, WA (US)

(72) Inventor: Leon Breckenridge, Spokane, WA (US)

(21) Appl. No.: 18/077,494

(22) Filed: Dec. 8, 2022

(51) Int. Cl. C12M 1/107; C12M 1/00; C12M 1/02; C12M 1/33; C12M 1/34

(52) U.S. Cl. CPC C12M 21/04; C12M 23/36; C12M 41/24; C12M 41/40; C12M 45/02; C12M 45/20

(57) ABSTRACT

Methods of keeping microorganisms working at maximum efficiency in an anaerobic digester to produce a renewable biogas, within a temperature range of 100 degrees F., preferably at a plus or minus of 0.5 degrees F., without adding heat to the mixture. Most preferably, the method includes premixing a batch of biomass including the microorganisms to form a uniform mixture, which also can include preheating the mixed biomass to approximately 100 degrees F. The digester operation identifies the rate that the mixture in the digester can be changed to a different mixture. Furthermore, the method can include separately collecting the CH₄ biogas and the CO₂ biogas in different chambers to meet the desired concentration of each these gases and can include removing from the biogas any sulfur containing gas and liquid water, and additionally separating lighter and/or heavier than water non-organic materials prior to them entering the digester.

### TECHNICAL FIELD

The present disclosure relates to methods of keeping microorganisms working at maximum efficiency in an anaerobic digester to produce a renewable biogas, and is generally applicable to the treatment of biomass in anaerobic processes. It is particularly applicable to methods of keeping microorganisms working at maximum efficiency in an anaerobic digester to produce a renewable biogas, but may be applied to other activated sludge process configurations with similar benefits, as described herein.

### BACKGROUND OF THE INVENTION

Nature has been converting biomass into fossil energy for billions of years. The result of this conversion is the coal, oil and natural gas that are extracted from the earth today. There is nearly an unlimited amount of energy received from the sun each day. Plants convert some of this sunlight into biomass via photosynthesis. This biomass can have an energy return of 100, or even 1,000 times as much as all of the energy necessary to plant, harvest, and move and convert it into renewable natural gas. Additionally, there are up to about six trillion microorganisms per gram in healthy soil. Many of these microorganisms inhabit the digestive systems of bovines (cattle) and have at least since the Bronze Age. These microorganisms are very robust. They can survive in very harsh environments from hot to far below freezing and pressures from high to below atmospheric. However, they are very sensitive to the many conditions required for producing biogas at a maximum rate.

### BRIEF DESCRIPTION OF THE DRAWINGS

Exemplary embodiments of the technology will become more fully apparent from the following descriptions and appended claims, taken in conjunction with the accompanying drawings. Understanding that these drawings depict only exemplary embodiments and are, therefore, not to be considered limiting of the scope of the technology, the exemplary embodiments will be described with additional specificity and detail through use of the accompanying drawings in which:

FIG. 1 is a schematic drawing of an AADS (Advanced Anaerobic Digester System) Digester Flow Schematic, according to an embodiment of the invention;

FIG. 2 is a schematic drawing of an AADS Schematic Receiving, Pretreatment and Storage, according to an embodiment of the invention;

FIG. 3 is a schematic drawing of an AADS Digester Infeed, according to an embodiment of the invention;

FIG. 3A is a schematic drawing of a portion of the AADS Digester Infeed Section as referenced in FIG. 3, according to an embodiment of the invention;

FIG. 4 is a schematic drawing of an AADS Dig. Inorganic Removal, according to an embodiment of the invention;

FIG. 5 is a schematic drawing of an AADS Insulation Bottom, according to an embodiment of the invention;

FIG. 6 is a schematic drawing of an AADS Digester Cross Section, according to an embodiment of the invention;

FIG. 7 is a schematic drawing of an AADS Dig. Top Insulation & Attachment, according to an embodiment of the invention;

FIG. 8 is a schematic drawing of an AADS Digester Side Insulation, according to an embodiment of the invention;

FIG. 9 is a schematic drawing of an AADS Digester Brew Crust Control, according to an embodiment of the invention;

FIG. 10 is a schematic drawing of an AADS Digester Piping and Support, according to an embodiment of the invention;

FIG. 10A is a schematic drawing of a portion of the AADS Digester Piping and Support, according to an embodiment of the invention;

FIG. 10B is a schematic drawing of a portion of the AADS Digester Piping and Support, according to an embodiment of the invention;

FIG. 11 is a schematic drawing of an AADS Digester Exit Brew Removal, according to an embodiment of the invention;

FIG. 11A is a schematic drawing of portion of the AADS Digester Exit Brew Removal, as referenced in FIG. 11, according to an embodiment of the invention; and

FIG. 12 is a schematic drawing of an AADS Digester Volume and Pressure Control, according to an embodiment of the invention.

Reference characters included in the above drawings indicate corresponding parts throughout the several views, as discussed herein. The description herein illustrates one preferred embodiment of the invention, in one form, and the description herein is not to be construed as limiting the scope of the invention in any manner. It should be understood that the above listed figures are not necessarily to scale and may include fragmentary views, graphic symbols, diagrammatic or schematic representations. Details that are not necessary for an understanding of the present invention by one skilled in the technology of the invention, or render other details difficult to perceive, may have been omitted.

### DETAILED DESCRIPTION OF SPECIFIC EMBODIMENTS

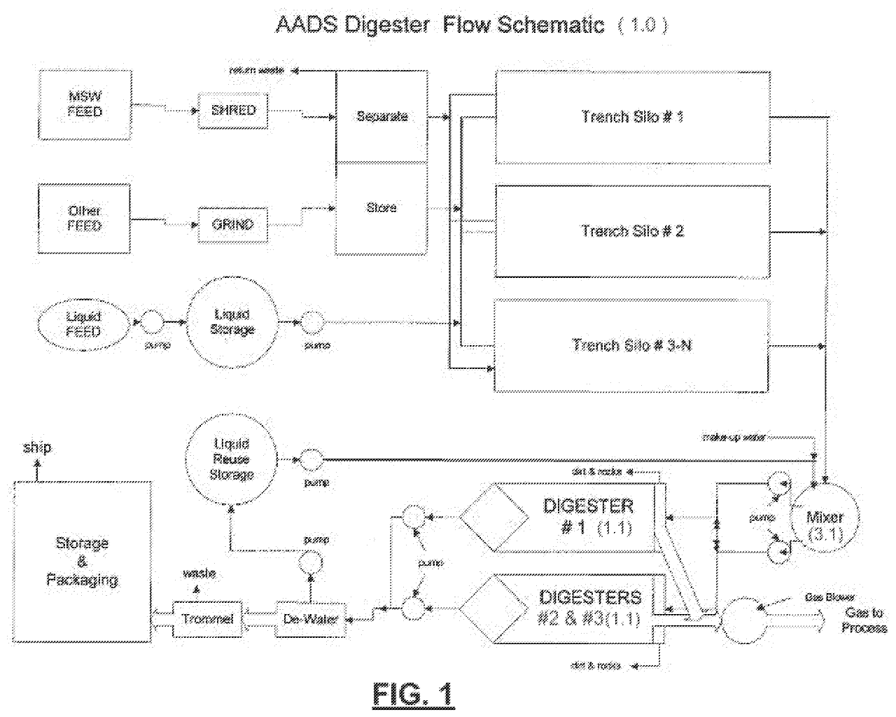

The foregoing description applies what we know about nature's microorganisms and explains how to convert much of the biomass into methane gas and CO₂, referred to herein as “energy convertors.” Exemplary embodiments of “methods of keeping microorganisms working at maximum efficiency in an anaerobic digester to produce a renewable biogas”, or alternatively referred to herein as the “method for production of a renewable bio-natural gas” or more simply the “anaerobic digester method of operation” 1.0, will be best understood by reference to FIGS. 1 through 12 included herewith, wherein like parts are designated by like numerals throughout. It will be readily understood that the components of the device, as generally described and illustrated in the figures herein, could be arranged and designed in a wide variety of different configurations. Thus, the following more detailed description of the embodiments of the apparatus, system, and method is not intended to limit the scope of the invention, as claimed, but is merely representative of exemplary embodiments of the technology. The following are a set of operational factors required to produce a “renewable natural gas” (RNG) from organic materials, referred to herein as a “renewable biogas” or more simply a “biogas”, utilizing natural or naturally occurring microorganisms:
a. These natural microorganisms work at maximum efficiency within a temperature range of 100 degrees F. plus or minus 0.5 degrees F.
b. Production of CH₄ and CO₂ occur at different stages in the process.
c. Early in the process, over 90% CH₄ by volume is produced in the biogas.
d. Late in the process, over 90% CO₂ by volume is produced in the biogas.
e. These natural microorganisms work at maximum efficiency when the gas molecule formed by the microorganism almost immediately (typically in a period of seconds) leaves the microorganism where it was produced.
f. These natural microorganisms also work at maximum efficiency when the pressure of the liquid mixture is at nearly a constant level, preferably under approximately 15 psig.
g. These natural microorganisms require a minimum carbon/nitrogen ratio to survive.
h. Organic materials typically have non-organic materials mixed with them.
i. Typically, most biomass is a mixture, containing fibers/straw, sand, gravel or small rocks, and also comprises thick liquids that may behave like glue, along with plastic-like materials that typically float or are relatively buoyant.

The present disclosure includes inventive methods of keeping these natural microorganisms working at a maximum efficiency, which preferably is held at a temperature of 100 degrees F., most preferably in a range of plus or minus 0.5 degrees F., and importantly preferably maintained without adding heat to the biomass and microorganism mixture while it is producing the biogas.

Additionally, the methods disclosed herein can include premixing a batch of biomass including the natural microorganisms, so the batch is a uniform mixture, and preferably includes preheating the mixed biomass to 100 degrees F. plus or minus degrees F.

The natural microorganisms acclimate to each specific and unique mixture of biomass feed stock. Changing that feed stock mixture too rapidly will make the microorganisms sick and require the system to be drained and start over in the same way the system was initially started.

Therefore, an anaerobic digester method of operation is herein proposed to identify the rate that the current mixture in the biogas producing chamber can be changed-out to a different mixture. Typically, this rate of change-out may be as short as approximately 5 days and as long as approximately 20 days. Due to this need the amount of different biomass for a given mixture needs to be of a sufficient amount to operate the biogas producing chamber, typically for many weeks.

Furthermore, the anaerobic digester method of the present invention can include separately collecting the CH₄ biogas and the CO₂ biogas in different chambers, each operated concurrently to meet the desired concentration of each these collected biogases.

Also, the anaerobic digester method of the present invention can additionally include controlling the pressure of the biogas so it is always above atmospheric pressure but never more than approximately ten inches of water column (approx. 0.36 pounds per sq. in.) above typical atmospheric pressure as measured at the site of the biogas producing chamber or anaerobic digester.

Also, the anaerobic digester method of the present invention can include removing any sulfur containing gases, and substantially all liquid water from the biogas.

Alternatively, the anaerobic digester method 1.0 separating lighter than water non organic materials from the biogas prior to these components entering a biogas producing chamber or “DIGESTER” 1.1 shown in FIG. 1, therefore, in an alternative embodiment of the present invention, the anaerobic digester method can include separating heavier than water non-organic materials from the organic biomass, before the biogas is produced.

The anaerobic digester method 1.0 can include feeding the prepared feed stock into the biogas producing chamber without a need for the use of, or employment of a valve, as noted in operational factors h. and i., listed above. Any conventional type of mechanical valve or similar functioning equipment element that is not especially smooth with an extraordinarily low roughness at both the inlet and outlet, will collect fibers and solids from the mixture. These fibers and solids generally will keep collecting until the inlet pie or the outlet pipes are plugged. Typically, there is also sand and gravel in the mix that will cause a valve to not close completely. A preferred embodiment of the anaerobic digester method of the present invention can also include removing the digested feed stock from the biogas producing chamber without the need for a valve. Furthermore, much of what is listed or described herein regarding the inlet is also applicable to the outlet of the biogas producing chamber.

Further referring to the attached FIGS. 1 through 12, the “Advanced Anaerobic Digester System” (AADS) titled “AADS Digester Flow Schematic” as shown in FIG. 1 is a typical layout of a site to produce renewable natural gas (RNG). Each actual layout will be site specific. For example, some sites may have Trench Silo's while others may not have any. Digester #1, Digesters #2 and Digester #3 (referenced as 1.1) are shown. Alternatively, there may be only one digester, or as many as ten, or more.

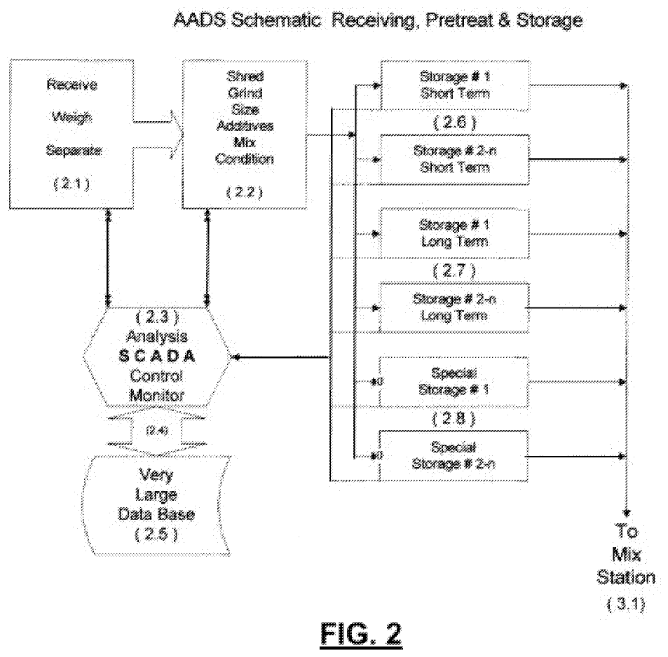

The titled “AADS Receiving, Pretreatment & Storage” schematic of FIG. 2 is an example of a typical layout for a site. This layout also will be site specific. This is only one example, and each site will be specific for the available feed stocks. With Receive, Weigh, and Separate steps 2.1, each site will have a method of measuring how much of each different type of feed stock is being received. Generally, it will need to be separated so it can be stored with similar feed stocks. Then, with Shred Grind, Size, Mix and Condition steps 2.2, each site will have a method of sizing and mixing the preferred amount of each selected input material. Most preferably, premixing the batch of biomass including microorganisms, is performed with the internal mixture of the pre-mixed batch of biomass, so that this internal mixture is substantially homogeneous. Analysis, SCADA, Control and Monitor process steps 2.3, and with a Very Large Database 2.5 and data interface 2.4 schematically illustrate in FIG. 2 there is a large amount of data and data processing needed to operate and control the digester system. Short Term Storage 2.6, Long Term Storage 2.7, and Special Storage 2.8 are examples of the typical material storage needed. A Mix Station 3.1 must be specifically designed for each site, with the design and configuration of the Mix Station considered conventional and well-known, very site specific, having many possible variations.

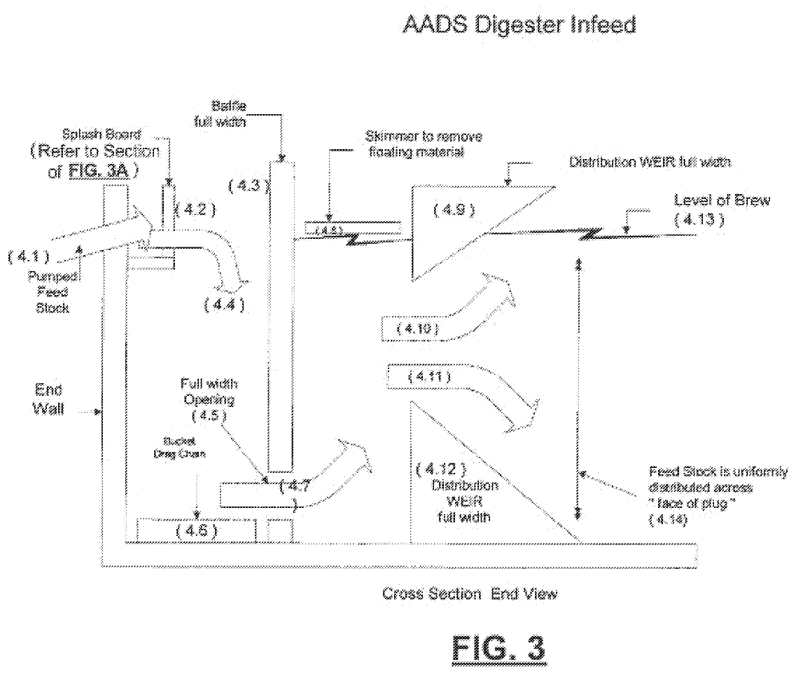

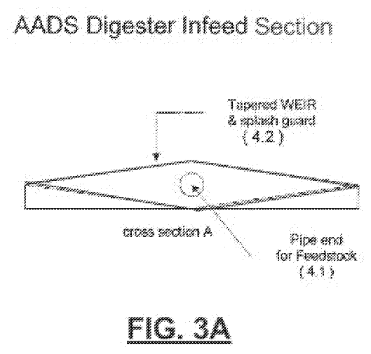

The titled “AADS Digester Infeed” schematic of FIG. 3 shows how the feed stock is slowed to a “crawl” so that heavier than water material will settle and lighter than water material will float. Preferably, a Pumped Feed Stock 4.1 as also detailed in FIG. 3A, is moved at a relatively high velocity and retained by a Splash Board 4.2 positioned across a full width of the digester, with a large opening across the digester so there is no velocity remaining at a splash board through-flow 4.4. A Full width Baffle 4.3 is positioned above the highest level of the brew within the digester. A Full width Opening 4.5 forces all of the infeeding brew or feed stock to enter the digester proper, near the bottom and all the way across in a baffle flow stream 4.7. A Bucket Drag Chain 4.6 collects and scraps the bottom of the digester to remove dirt and heavier than water materials so then can be removed. A Skimmer to remove floating material 4.8 allows all of these non-digestible constituents to be removed. A Distribution Weir 4.9 and 4.12 assures that the feed stock 4.10 and 4.11 is uniform from one side to the other of the digester. Most preferably, a Level of Brew 4.13 is maintained within a few inches of a constant level. The Feed Stock is uniformly distributed across a “face of plug” 4.14, from top to bottom, so it is moving through the digester as a “plug.”

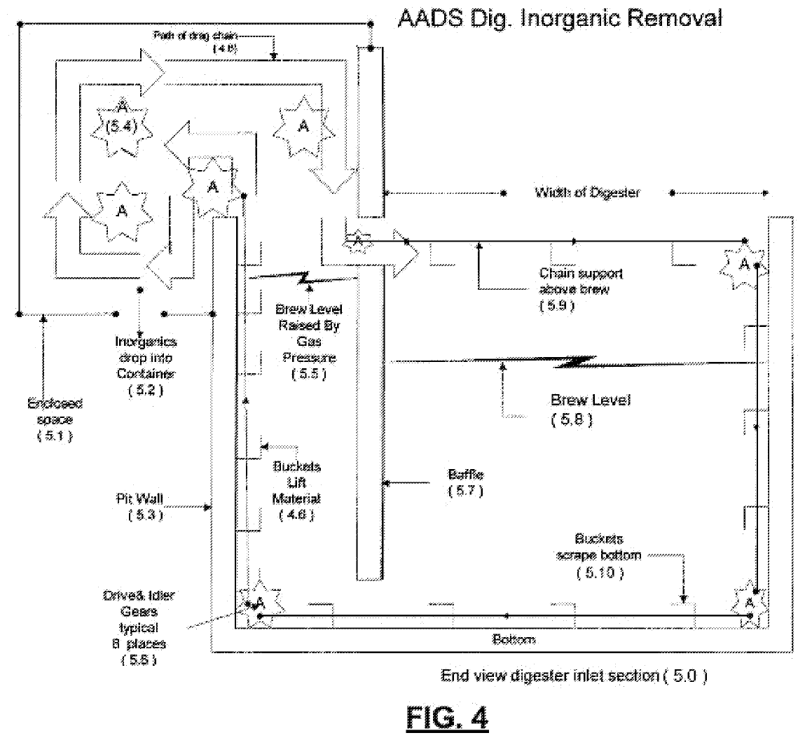

The titled “AADS Digester Inorganic Removal” schematic of FIG. 4 shows how the feed stock that is heavier than water is removed. This is related to the AADS Digester Infeed FIG. 3, including drag chain 4.8 and shows more of the functions. Preferably, this is all a part of an Enclosed space 5.1, so only Inorganics drop into Container 5.2 from the brew feed stock. Pit wall 5.3 includes both sides and bottom of the digester inlet section 5.0, as shown. These also provide the support for the drag chain included in a Buckets that scrape bottom 5.10 of the digester, and Idler gears 5.4 and 5.6 preferably for a total of eight places, designated “A” therein. This action scrapes the side and bottom of the inlet end for the feed stock and includes a Baffle 5.7 and Chain support above brew 5.9, while 5.8 illustrates where the Brew Level would likely expected to be.

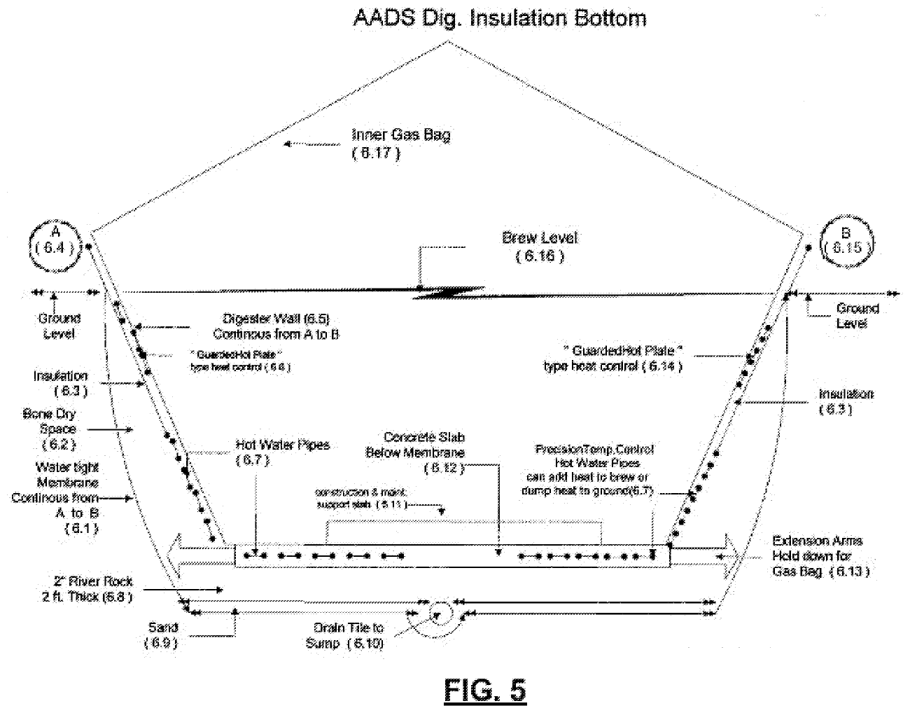

The titled “AADS Digester Insulation” schematic of FIG. 5 shows how to prevent any outside condition that is colder than the brew or digesting feed stock from cooling off the brew within the digester. A Water tight Membrane 6.1 helps assure a Bone Dry Space 6.2 immediacy within the Water tight Membrane to stay dry, so it will reduce the amount of heat that could enter or leave the brew across the brew walls. Insulation 6.3 is the primary method of keeping the brew at constant temperature. The Digester Wall 6.5 is water tight. Reference points “A” 6.4 and “B” 6.15 therein, show the Digester Wall, Insulation and Water tight membrane preferably extending some distance above a Ground Level. A “Guarded Hot-Plate” 6.6 is a preferable method employed for the anaerobic digester method 1.0, where a Hot Water Pipes 6.7 and 6.14 are maintained at a temperature a fraction of a degree above the desired brew temperature. The weight of the brew container is supported by approximately two inches of River Rock 6.8 or similar typical material that will hold the weight of the brew enclosure without deforming. At its two inches of thickness, the River Rock has only “point contact”, so as long as it stays dry it is a good insulator. Preferably, Sand 6.9 is placed under the rock to protect the water tight liner. Also, a Drain Tile 6.10 to Sump is installed in the event liquid gets into this space near the liner. During construction and maintenance a support slab 6.11 is used to protect the bottom of the container. There is a Concrete Slab 6.12 below the membrane, which also provides support for the Extension Arms 6.13 that hold down an Inner Gas Bag 6.17. Brew Level 6.16 illustrates the level of the brew within the Inner Gas Bag, as also illustrated in FIG. 5.

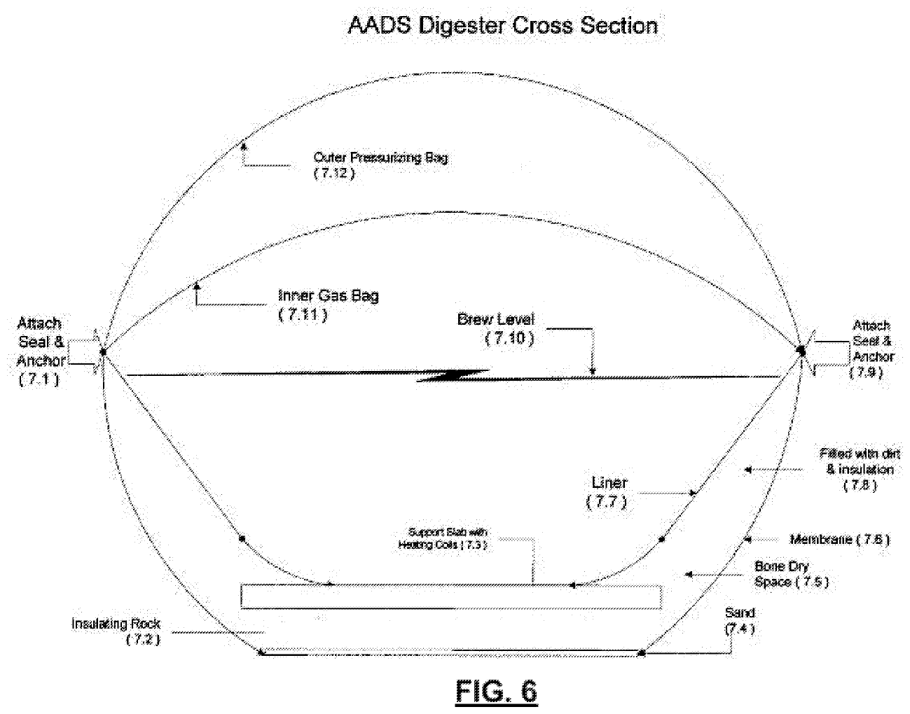

The titled “AADS Digester Cross Section” schematic of FIG. 6 details the insulation in FIG. 5 to be sure there are no leaks and prevent “cold” from getting to the brew or more properly to prevent heat from escaping from the digester. A feature of the digester system of the present invention is that the Outer Pressure Bag 7.12 is maintained at a constant pressure and expected to withstand winds of over 50 mph. The Inner Gas Bag 7.11, Liner 7.7, and outer bag are sealed by Attach Seal and Anchor 7.1 and 7.9. Insulating Rock 7.2 and Sand 7.4 are previously discussed herein, in relation to River Rock 6.8 and Sand 6.9. Support Slab with heating Coils 7.3 is discussed in 6.12. Bone Dry Space 7.5 is also discussed in 6.2. Membrane 7.6 is waterproof and prevents moisture from the Filled with Dirt and Insulation 7.8 dry space below grade, noting that Brew level 7.10 shown in FIG. 6 only illustrates where it may likely be.

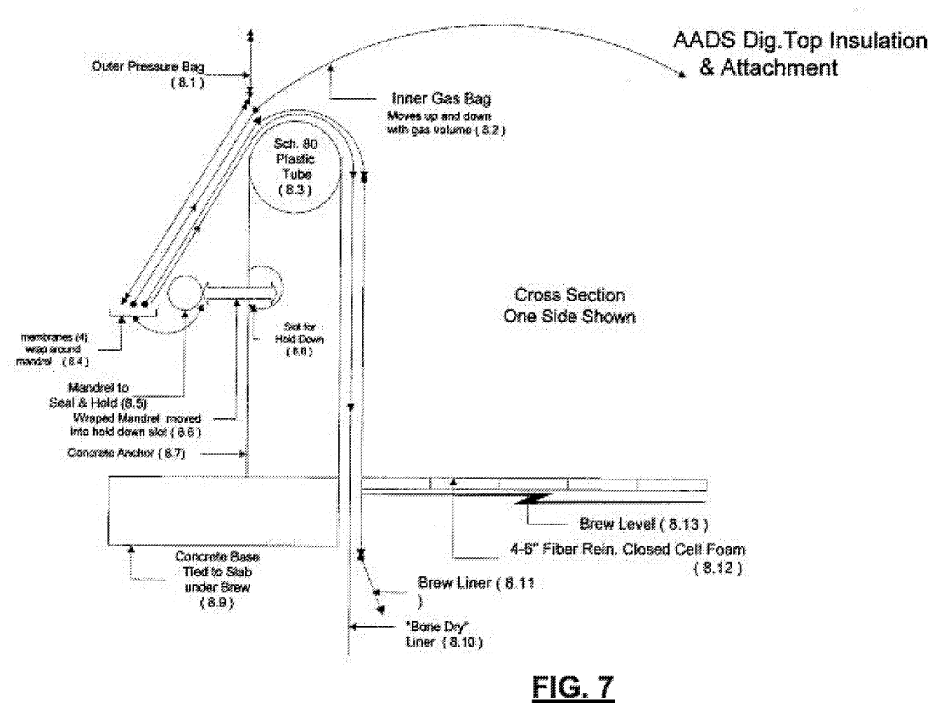

A preferred method for titled “AADS Dig. (or Digester) Top Insulation & Attachment” is shown schematically in FIG. 7, which includes additional details associated with FIG. 5. Outer Pressure Bag 8.1 is at a constant pressure and fully inflated. Inner Gas Bag 8.2 moves up and down depending on amount of biogas in the bag. Tube 8.3 is a larger diameter tube of approximately eighteen inches or more in diameter, and preferably a Standard Sch. 80 type of Plastic “PVC” Pipe supported along its full length by Concrete Anchor 8.7. There are four membranes 8.4 that include a “Bone Dry” Liner 8.10 and a Brew Liner 8.11 that both wrap around Mandrel 8.5, which after the membranes are at tension the Mandrel is moved into a Hold Down Slot 8.6 and a Slot for Hold Down 8.8. All of these components of FIG. 7 are supported by a Concrete Base 8.9. Most preferably, a Closed Cell Foam 8.12 floats upon the brew to prevent heat loss, and it is notable that a Brew level 8.13 is shown as only an illustration of where it might be located.

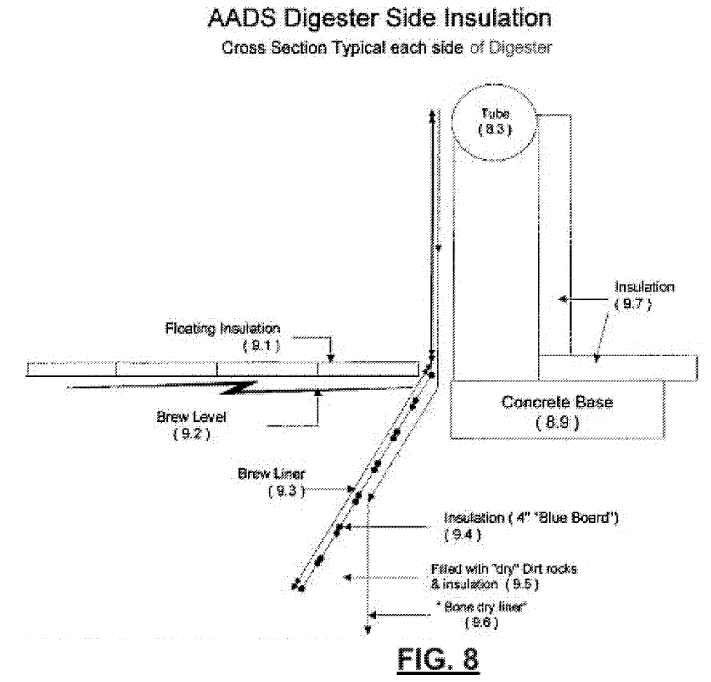

The titled “AADS Digester Side Insulation” schematic of FIG. 8, shows additional details associated with FIG. 5. Specifically, FIG. 8 shows the opposite side of what is shown in FIG. 7, except the only anchor point is on the side shown in FIG. 7. FIG. 8 also shows a Floating Insulation 9.1, a Brew Level 9.2, a Brew Liner 9.3, Insulation 9.4 that is most preferably a conventional four inch thick “Blue Board” closed cell foam, filled with “dry” Dirt, rocks and insulation 9.5, a Bone Dry Liner 9.6, and Insulation 9.7.

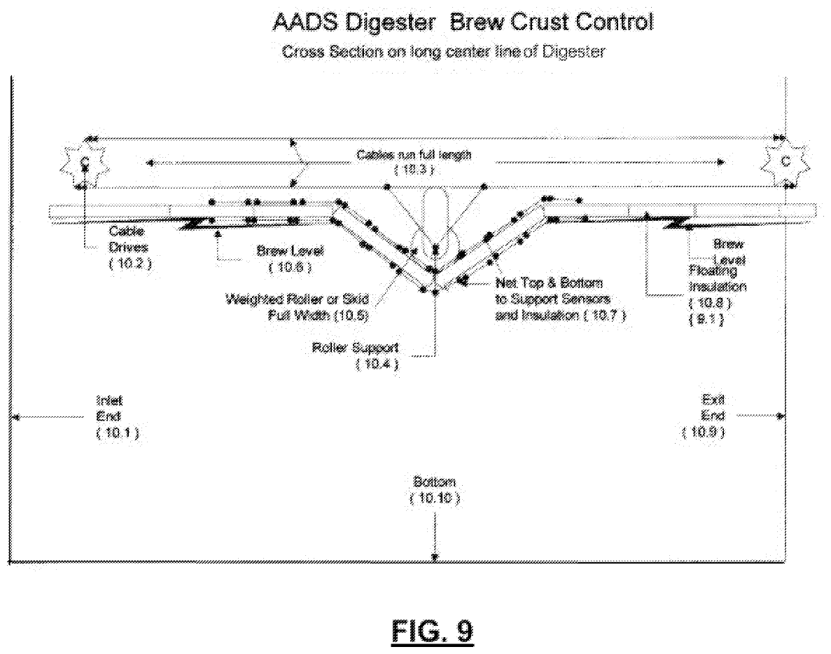

The titled “AADS Digester Brew Crust Control” schematic of FIG. 9, addresses any crust that may form at the surface of the brew at a Brew Level 10.6, for the purpose of keeping any particles that may be on the surface from forming a crust and keeps them wet. A Cable Drive 10.2 moves a Weighted Roller or Skid 10.5 that travels the full width of the digester, back and forth from one end to the other by Cables 10.3. The feed stock moves from an Inlet End 10.1 to an Exit End 10.9. There is a top Cable 10.3 that is contentious that also runs underneath and pulls the Roller Support 10.4 from one end to the other and then reverses and returns back to the other end. The weighted roller 10.5 travels along the brew level, at its top surface, which may fall as far as the Bottom 10.10 brew level. Most preferably, a Net 10.7 also runs the full length of the brew, to keep the sensors and Floating Insulation 10.8 in place. The Floating Insulation is also shown in FIG. 8 as reference 9.1.

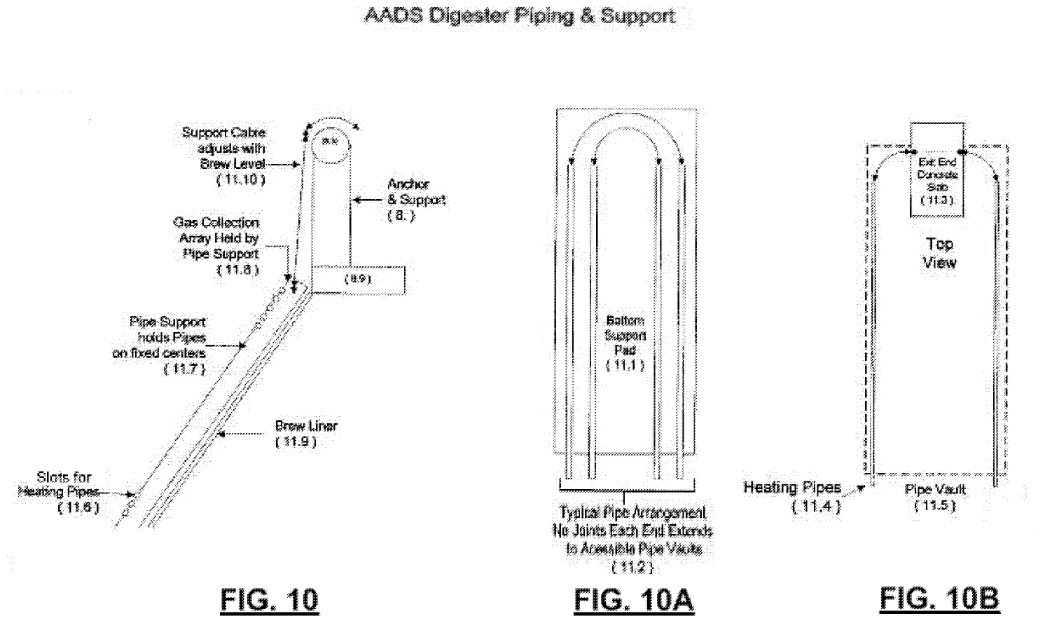

The titled “AADS Digester Piping & Support” schematic of FIG. 10 shows details associated with previously discussed FIG. 5, and also shows how to prevent any outside condition that is colder than the brew or feed stock within the digester from extracting heat from the brew. A Bottom Support Pad 11.1 is illustrated in the detail of FIG. 10A, but with only two of the preferably many possible pipes, including a Pipe Vault 11.2 and 11.5 are as detailed in FIGS. 10A and 10B, respectively. An Exit End Concrete Slab 11.3 shown in detail FIG. 10B included a multiple of Heating Pipes 11.4 and a Pipe Vault 11.5. Additionally, Slots for Heating Pipes 11.6, Pipe Support 11.7, a Gas Collection Array Held by Pipe Support 11.8, a Brew Liner 11.9, and a Support Level 11.10 are all as previously detailed in FIG. 7, and with the Outer Pressure Bag 8.1 and the Concrete Base 8.9, also as shown in FIG. 10.

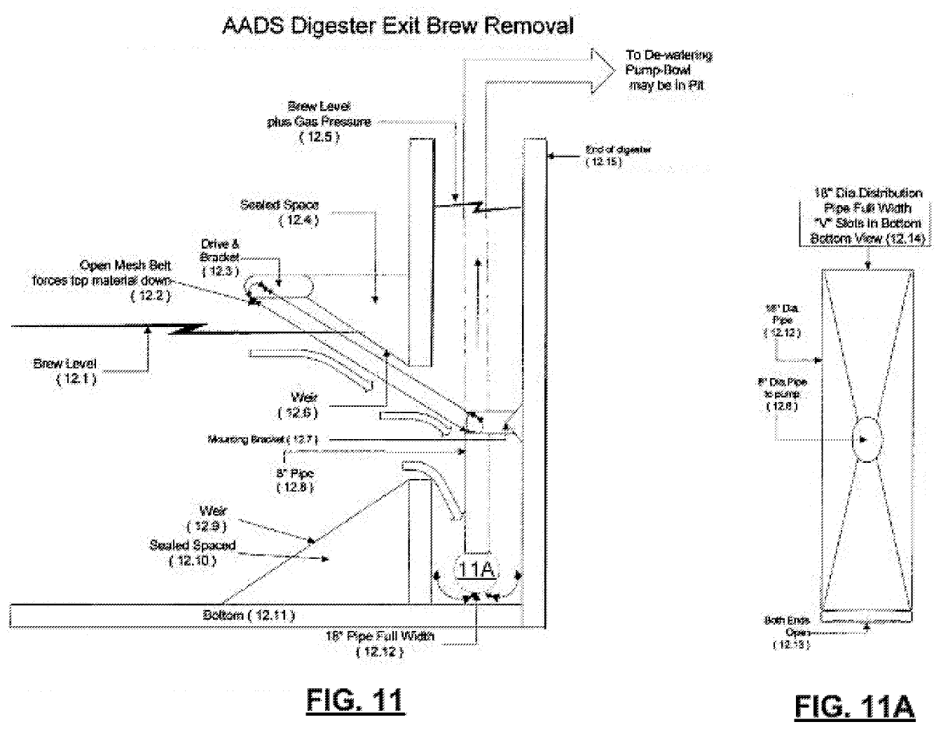

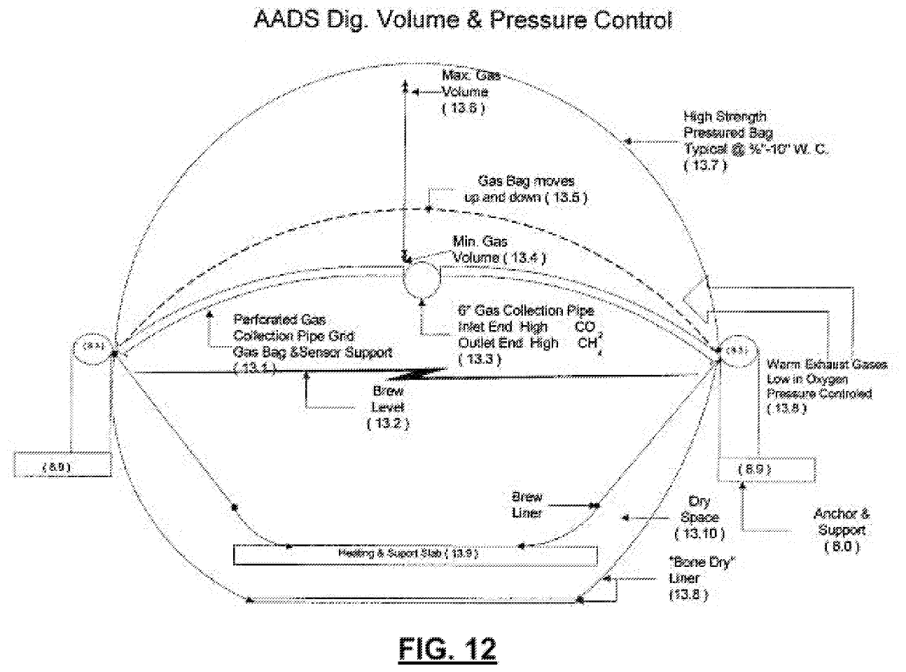

The titled “AADS Digester Exit Brew Removal” schematic of FIG. 11, also refers to similar details shown in FIG. 3 which relates to details of the infeed. Material lighter than water will float or be relatively buoyant. FIG. 12 shows how the brew or digested feed stock is removed without the use of valves. Brew level 12.1 is an illustration of the typical brew level within the digester. Open Mesh Belt 12.2 is driven by a Drive & Bracket 12.3. This action forces the floating material into the space where the discharge pump is located. This Sealed Space 12.4 and a First Weir 12.6 are the supports for the Drive & Bracket 12.3. In the space between the Brew Level 12.1 and a Brew Level plus Gas Pressure 12.5, the Brew Level will be higher by the amount of biogas pressure. A Mounting Bracket 12.7 is for the other end of the Mesh Belt. The discharge pipe 12.8 is preferably an eight inch diameter pipe. Also most preferably, there is a Second Weir 12.9 with a Sealed Space 12.10 attached to the Bottom 12.11 of the digester. Full Width Pipes 12.12 and 12.13 each preferably have approximately an eighteen inch diameter and are detailed in Bottom View 12.14, and additionally with an End of Digester 12.15 as detailed in FIG. 11A, which also is referenced in FIG. 11.

The titled “AADS Digester Volume & Pressure Control” schematic of FIG. 12 shows a number of details that are also referenced on FIG. 7, including therein labeled Outer Pressure Bag 8.1, Inner Gas Bag 8.2, and the Concrete Base 8.9. FIG. 12 further details how the pressure of the biogas is always above a typical atmospheric pressure but never more than about ten inches of water column (approx. 0.36 pounds sq. in.) above the typical atmospheric pressure as measured at the site of the digester. Perforated Gas Collection Pipe 13.1 is above the maximum Brew Level 13.2. These pipes are connected to a six inch gas collection pipe 13.3. Min gas volume 13.4 is at this point. The Gas Bag (or Inner Gas Bag) 13.5 moves up and down in this space. Max. Gas Volume 13.6 is achieved when the Inner Gas Bag reaches the High Strength Pressure Bag (or Outer Pressure Bag) 13.7. The Bone Dry Liner 13.8, Heating and Support Slab (or Concrete Base) 13.9, and Dry Space 13.10 are also detailed in FIG. 7.

For this Detailed Description of Specific Embodiments, the terms “connected”, “attached”, “coupled”, and “mounted” refer to any form of interaction between two or more elements, including mechanical, electrical, magnetic, electromagnetic, fluid, and thermal interaction. Two components may be functionally coupled with or to each other, even though they are not in direct contact with each other.

Also, the terms “approximately” or “approximate” are employed herein throughout, including this detailed description and the attached claims, with the understanding that is denotes a level of exactitude commensurate with the skill and precision typical for the particular field of endeavor, as applicable.

Additionally, the terminology used in this Detailed Description of Specific Embodiments is to be interpreted according to ordinary and customary usage in the field of the invention as exemplified in the pertinent U.S. and International Patent Classification Codes, and equivalent codes in other patent classification systems.

The word “embodiment” is used herein to mean “serving as an example, instance, or illustration.” Any embodiment described herein is not necessarily to be construed as preferred or advantageous over other embodiments. While the various aspects of the embodiments are presented in drawings, the drawings are not necessarily drawn to scale.

Additionally, reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above Detailed Description of Specific Embodiments includes the referenced figures and following claims, and is more simply referred to herein as the “description” or the “disclosure”. In this description, various features are sometimes grouped together in a single embodiment, figure, or written explanation or description thereof for the purpose of streamlining this disclosure. However, this method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this description are hereby expressly incorporated into this description and disclosure, with each claim standing on its own as a separate embodiment. The description includes all permutations of the independent claims with their dependent claims.

In compliance with the statutes, the invention has been described in language more or less specific as to structural features and process steps where applicable. While this invention is susceptible to embodiment in different forms, the specification illustrates preferred embodiments of the invention with the understanding that the present disclosure is to be considered an exemplification of the principles of the invention, and the disclosure is not intended to limit the invention to the particular embodiments described. Those with ordinary skill in the art will appreciate that other embodiments and variations of the invention are possible, which employ the same inventive concepts as described above. Therefore, the invention is not to be limited except by the following claims, as appropriately interpreted in accordance with the doctrine of equivalents.

The following is claimed:

1. A method of keeping microorganisms working at maximum efficiency in an anaerobic digester to produce a biogas, the anaerobic digester method comprising the steps of:
a. maintaining an internal mixture within the anaerobic digester at a temperature of approximately 100 degrees F.; and
b. thermally isolating the internal mixture within the anaerobic digester to substantially stop a flow of heat to or from the internal mixture, during the production of the biogas.

2. The anaerobic digester method of claim 1, additionally including the step of:
c. premixing a batch of biomass including microorganisms to form the internal mixture as a pre-mixed batch of biomass, so that said internal mixture is substantially homogeneous.

3. The anaerobic digester method of claim 2, additionally including the step of:
d. preheating the pre-mixed batch of biomass including microorganisms to 100 degrees F.

4. The anaerobic digester method of claim 2, additionally including the step of:
e. slowly changing out the pre-mixed batch of biomass including microorganisms within the anaerobic digester, to avoid making the microorganisms sick.

5. The anaerobic digester method of claim 1, additionally including the step of:
c. separately collecting a CH4 biogas and a CO2 biogas each in a different and separate chamber of the anaerobic digester, to meet a desired concentration of the CH4 biogas and a desired concentration of the CO2 biogas.

6. The anaerobic digester method of claim 1, additionally including the step of:
c. controlling the pressure of the biogas contained within the anaerobic digester so the pressure of the biogas within the anaerobic digester is always above a site atmospheric pressure but never more than approximately 10 inches of water column (or approximately 0.36 pounds per sq. in.) above the site atmospheric pressure.

7. The anaerobic digester method of claim 1, additionally including the steps of:
c. removing from the biogas produced by the anaerobic digester, any of a sulfur containing gas components; and
d. removing any of a liquid water from the biogas produced by the anaerobic digester.

8. The anaerobic digester method of claim 2, additionally including the step of:
d. separating a lighter than water non-organic material from the pre-mixed batch of biomass, prior to the pre-mixed batch of biomass entering the chamber of the anaerobic digester, where the biogas is produced.

9. The anaerobic digester method of claim 2, additionally including the step of:
d. separating a heavier than water non organic material from the pre-mixed batch of biomass, prior to the pre-mixed batch of biomass entering the chamber of the anaerobic digester, where the biogas is produced.

10. The anaerobic digester method of claim 1, additionally including the step of:
c. feeding the prepared feed stock into the biogas producing chamber without employing a valve for the removal of the digested feed stock.

11. The anaerobic digester method of claim 1, additionally including the step of:
c. removing a digested feed stock from the biogas producing chamber without employing a valve for the removal of the digested feed stock.

12. The anaerobic digester method of claim 1, wherein the step of maintaining the internal mixture within the anaerobic digester at a temperature of approximately 100 degrees F., further includes maintaining the internal mixture within the anaerobic digester at a temperature of 100 degrees F. plus or minus 0.5 degrees F.

13. The anaerobic digester method of claim 2, wherein the step of preheating the pre-mixed batch of biomass further includes preheating the pre-mixed batch of biomass including microorganisms to 100 degrees plus or minus 0.5 degrees F.
